(19) United States
(12) Patent Application Publication
(10) Pub. No.: US 2019/0282490 A1
(43) Pub. Date: Sep. 19, 2019

(54) Nopal Cactus infused Natural Deodorant

(71) Applicant: Caroline J Braglia, Oxford, CT (US)

(72) Inventor: Caroline J Braglia, Oxford, CT (US)

(21) Appl. No.: 16/049,945

(22) Filed: Jul. 31, 2018

Related U.S. Application Data
(60) Provisional application No. 62/642,172, filed on Mar. 13, 2018.

(51) Int. Cl. A61K 8/9789; A61K 8/25; A61K 8/9794; A61K 8/92; A61K 8/98; A61K 8/73; A61Q 15/00

(52) U.S. Cl. CPC A61K 8/9789; A61K 8/25; A61K 8/9794; A61K 8/0229; A61K 8/987; A61K 8/732; A61Q 15/00; A61K 8/922

(57) ABSTRACT

A topical deodorant composition comprising: Organic refined Shea butter; Organic Nopal Cactus; an ingredient selected from the group consisting of Sodium bicarbonate or Food Grade Diatomaceous Earth; Arrowroot starch; Organic Cocoa butter; Organic Mango Butter; Organic Carnauba Wax; Organic Bees Wax; Organic Coconut oil; Organic Apple Cider Vinegar; and pure essential oil extracts. A method of applying a topical deodorant composition of unrefined Shea butter, an ingredient selected from the group consisting of Sodium bicarbonate and Food Grade Diatomaceous Earth, Arrowroot Starch, Cocoa Butter, Mango Butter, pure essential oil; Nopal Cactus, Coconut oil, Apple Cider Vinegar, and optionally, Carnauba wax or Beeswax, said method comprising the step of applying said topical deodorant composition to an area of skin. A process of manufacturing a topical deodorant composition, said process comprising: mixing unrefined Shea butter, cocoa butter, coconut oil and/or mango butter into a composition; heating said composition over hot water at approximately 100° F. to 120° F.; stirring said composition until said composition is melted; adding arrowroot starch and sodium bicarbonate into said composition; stirring and heating said composition over hot water at about 100° F. to about 120° F. until said Arrowroot starch and said sodium bicarbonate are completely dissolved; adding in Nopal Cactus after the composition has been completely dissolved and combining the Nopal Cactus with the composition until both the Nopal Cactus and the composition are evenly distributed throughout thereby producing the final composition.

### CROSS-REFERENCES

This patent application claims the benefit of U.S. Provisional Patent Application No. 62/642,172 by inventor Caroline Braglia entitled “Nopal Cactus infused Natural Deodorant,” filed on Mar. 13, 2018, and which provisional application is fully incorporated by reference herein.

### TECHNICAL FIELD

The present invention relates to deodorant, and more specifically to a natural deodorant comprising Nopal Cactus.

### BACKGROUND

In the current marketplace, there are a wide variety of deodorants and antiperspirants intended to block axillary perspiration and mask odors. Most of these deodorants contain synthetic substances such as carcinogenic parabens, aluminum salts that can disrupt endocrine processes and accumulate in tissues of the body, triclosan, a well-known pesticide, as well as Triethanolamine (TEA) and Diethanolamine (DEA) which are ammonia compounds that can damage kidneys and liver when absorbed. The combination and long term use of these irritating substances can have potentially damaging effects to the user. As an increasing number of studies demonstrate some long term dangers of the use of these commercial antiperspirants and deodorants, these companies continue to manufacture and market stronger and more potent versions of themselves, some are even encouraging consumers to apply before bed to increase absorption overnight by blocking natural bodily processes and use more of their product upon wakening. This ultimately makes more money for the companies that have complete disregard for health.

Thus there is a need for a deodorant that overcomes the above listed and other disadvantages.

### SUMMARY OF THE INVENTION

The invention relates to a topical deodorant composition comprising: Organic refined Shea butter; Organic Nopal Cactus; an ingredient selected from the group consisting of Sodium bicarbonate or Food Grade Diatomaceous Earth; Arrowroot starch; Organic Cocoa butter; Organic Mango Butter; Organic Carnauba Wax; Organic Bees Wax; Organic Coconut Oil; Organic Apple Cider Vinegar; and pure essential oil extracts.

The invention also relates to a method of applying a topical deodorant composition of unrefined Shea butter, an ingredient selected from the group consisting of Sodium bicarbonate or Food Grade Diatomaceous Earth, Arrowroot Starch, Cocoa Butter, Mango Butter, pure essential oil; Nopal Cactus, Coconut oil, Apple Cider Vinegar, and optionally, Carnauba wax or Beeswax, said method comprising the step of applying said topical deodorant composition to an area of skin.

In addition, the invention relates to a process of manufacturing a topical deodorant composition, said process comprising: mixing unrefined Shea butter, cocoa butter, coconut oil and/or mango butter into a composition; heating said composition over hot water at approximately 100° F. to 120° F.; stirring said composition until said composition is melted; adding arrowroot starch and sodium bicarbonate into said composition; stirring and heating said composition over hot water at about 100° F. to about 120° F. until said arrowroot starch and said sodium bicarbonate are completely dissolved; adding in Nopal Cactus after the composition has been completely dissolved and combining the Nopal Cactus with the composition until both the Nopal Cactus and mixture are evenly distributed throughout thereby producing the final composition.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 shows a table of ingredients in the formulation along with their percentage composition including a permissible range; and

FIG. 2 is a flowchart showing one embodiment of manufacturing the deodorant;

FIG. 3 is a flowchart that continues from FIG. 2; and

FIG. 4 is a flowchart showing one embodiment of using the deodorant.

### DETAILED DESCRIPTION OF THE INVENTION

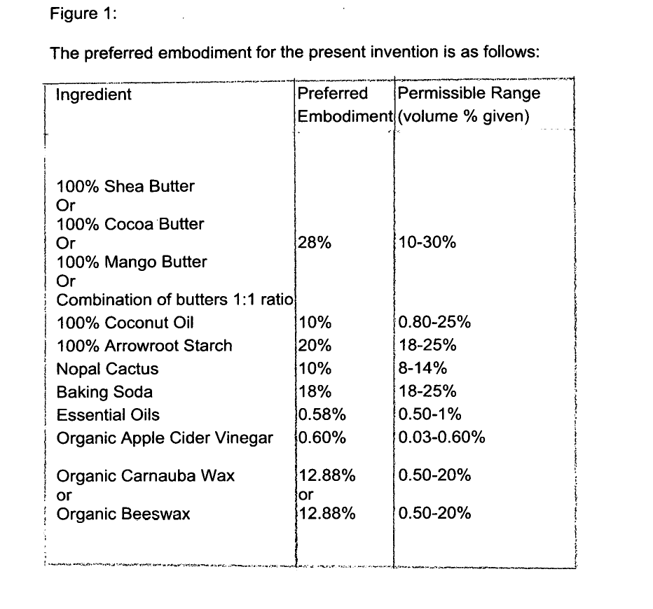

FIG. 1 shows a table of ingredients in the formulation along with their percentage composition including a permissible range.

Our product base is selected by the consumer from a choice of Organic Shea Butter, Organic Mango Butter or Organic Cocoa Butter. The consumer may also select a combination of all three butters. The butter(s) are melted down in double boiler glass container and blended with Organic Coconut Oil. The temperature of the water in the double boiler may be about 100° F. to about 120° F. The composition is stirred. Once melted completely into a liquid, Arrowroot Starch is then added along with the odor neutralizing selection of either Sodium Bicarbonate and Organic Apple Cider Vinegar or Diatomaceuous Earth. The composition is then stirred until all added powders have dissolved and the Organic Apple Cider Vinegar has been uniformly distributed into the liquefied formula. The Nopal Cactus is added in the form of a pure dried powder to the formula. It is folded in until dissolved and the color returns to a uniform lighter shade. The composition is then removed from the double boiler and the formulation is then cooled until 90% hardened in a cool room then whipped for approximately three minutes with a mixer until the product is light, creamy and easily spreadable. It is removed from the glass bowls and inserted into a narrow nozzle dispenser. The majority of the composition may be dispensed into glass jars then gently packed down until the jar is full. An Essential Oil or a combination of Essential Oils may be added to the formulation as well as the lid of the jar and immediately placed onto the jar of our natural deodorant formula to allow the infusion of the oil to infiltrate the composition providing a desired aroma as well as a specific health promoting benefit(s) from the chosen essential oil. Further, the processing uses only glass materials to obtain the least chemical contamination possible (the only plastic material is the consumer's selection of a deodorant stick applicator, which is BPA free and only poured into the applicator at a time where the butters/oils have cooled significantly to keep any chemical leeching to a minimum). From the glass jars, the composition is intended to be dispensed into the axilla with a wooden or a plastic applicator of the consumer's choice. Some consumers prefer to use their own hands to apply this composition to their underarms-application is preferential to consumer. We also offer a BPA free plastic deodorant stick for use with our more stable butter, Organic Cocoa Butter. This is a standard deodorant stick applicator that rolls up as needed.

The present invention contains no volatile silicones, nonpolar hydro-carbonic liquids, aluminum, alcohol, triclosan, parabens, pthalates, “fragrances” or propylene glycol. The ingredients utilized in this invention were carefully selected for their desirable health boosting properties like their antimicrobial actions, disruptions to carcinogenic pathways, pH balancing abilities, anti-inflammatory agents to reduce and prevent skin irritations from shaving, razor burn or ingrown hair prevention along with repairing nicks and cuts, absorbing perspiration, as well as the addition of phytochemicals and anti-oxidants which are excellent at combating free radicals. The ingredients chosen promote and enhance health. They assist with odor control by reducing unwanted bacteria (including Staphylcoccus, MRSA) that cause foul odors as well as controlling perspiration by restoration of homeostasis. They inhibit negative effects on the beneficial flora found naturally in healthy skin to maintain health and minimize any possibility of infection from more harmful pathogens. When probiotic numbers are higher than pathogenic numbers in the stratabiome, less odor is produced in the form of thioalcohols. This, in turn, causes a person's body odor to become more neutral and less offensive.

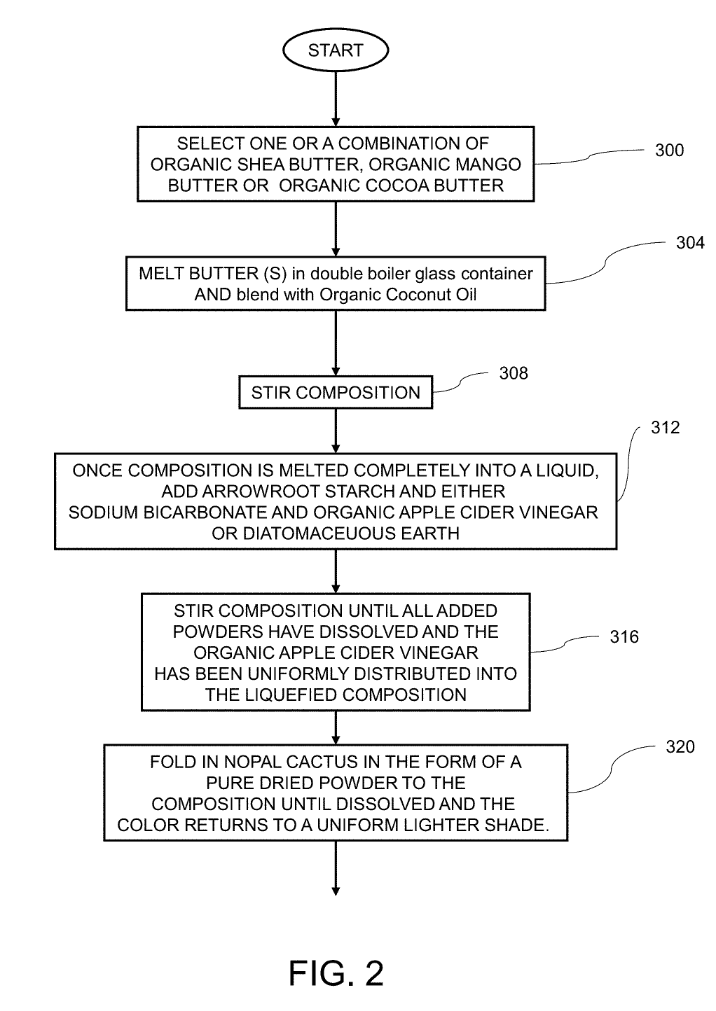

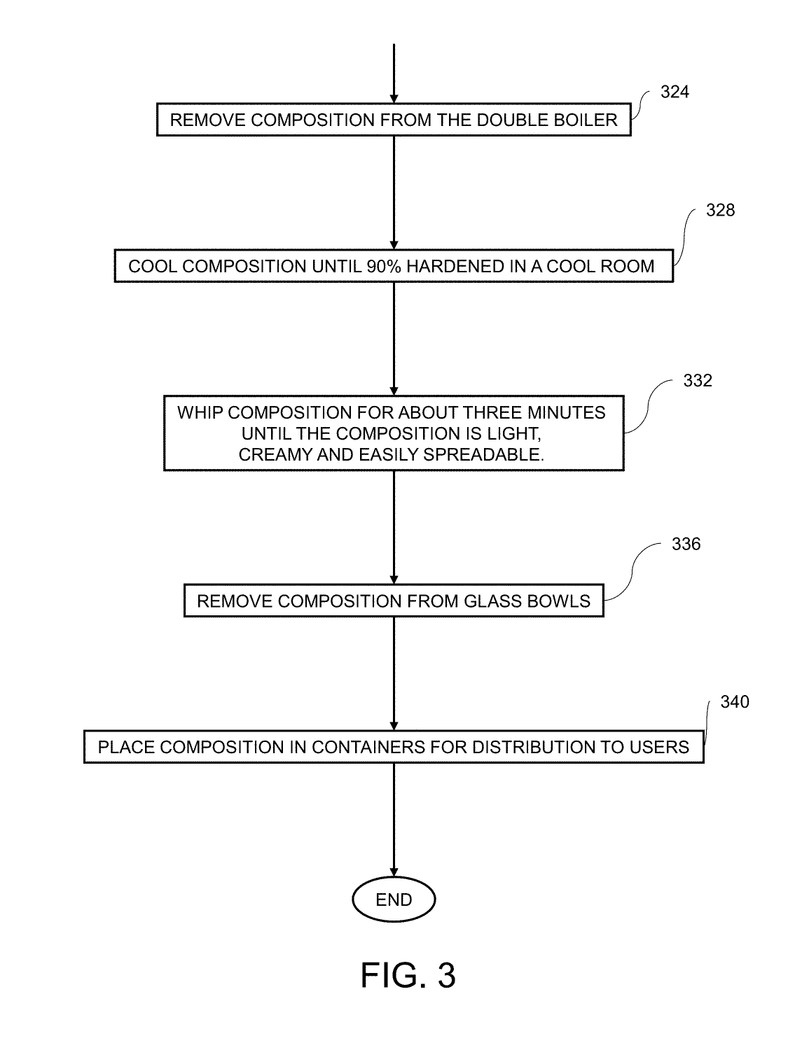

FIGS. 2 and 3 is a flowchart illustrating one method of making the nopal cactus infused natural deodorant. At act 300, the manufacturer selects one or a combination of organic shea butter, organic mango butter and organic cocoa butter. At act 304, the manufacturer melts the single butter or combination of butters in a double boiler glass container and blends the butter(s) with organic coconut oil. At act 308, the composition is stirred. At act 312, once the composition is melted completely into a liquid, arrowroot starch is added along with either sodium bicarbonate and organic apple cider vinegar on the one hand, or diatomaceuous earth. At act 316, the composition is stirred until all added powders have dissolved and the organic apple cider vinegar has been uniformly distributed into the liquefied composition. At act 320, Nopal Cactus in the form of a pure dried powder is folded into the composition until dissolved and the color returns to a uniform lighter shade. At act 324, the composition is removed from the double boiler. At act 328, the composition is cooled until the composition achieves a 90% hardness in a cool room. At act 332, the composition is whipped for about three minutes until the composition is light, creamy and easily spreadable. At act 336, the composition is removed from the glass bowls. At act 340 the composition is placed in containers for distribution to users. Act 340 includes placing the final composition in a glass jar, loading the final composition as a solid stick into a dispenser; and/or placing the final composition in a squeezable tube. Other means of distributing the composition to end users may also be included.

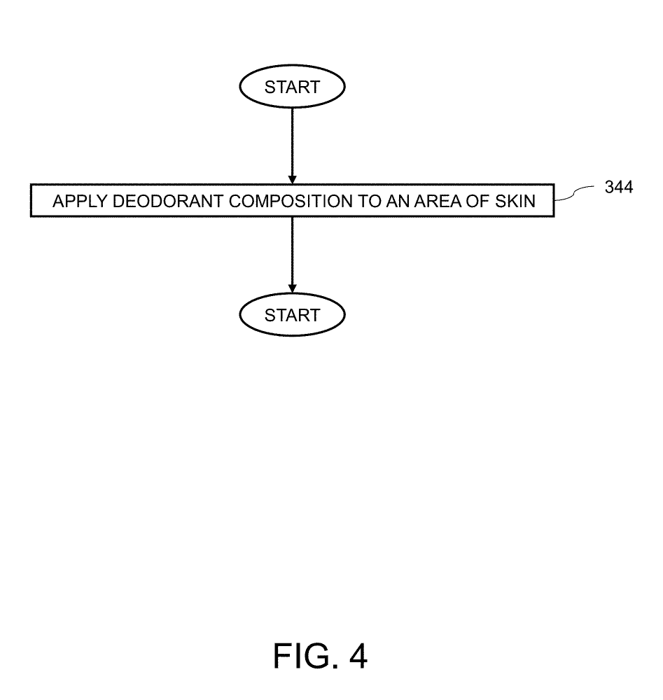

FIG. 4 is a flowchart showing how one may use the disclosed deodorant. At act 344, the user applies the deodorant composition to an area of his or her skin. This may include applying it via their fingers, or using a tool such as a wooden stick, or applying the deodorant if it is formed into a solid stick, and rubbing the solid stick on the user's skin.

With respect to the above ingredients, each performs an important role in producing an optimal deodorant which is effective, does not cause harm and promotes healthy skin. Coconut oil has been chosen because it is high in essential fatty acids as well as contains many antimicrobial properties and skin replenishing factors. It is also preferred as an ingredient for its malleable properties, makes our product easy to whip and apply as well as helps to control the stability of our stick options. Coconut oil is a well-known nutrient for the skin (body and hair) in that it contains high levels of antioxidants, has a light and sweet natural fragrance and is easily absorbed into the skin to reduce any residue or greasy feel.

Organic Shea Butter has been our original butter base for our deodorant due to its texture-thick and solid at room temperature which enables it to be used in a stick form as well as ajar. Shea butter is highly emollient as well as rich in anti-inflammatory and anti-oxidant properties. Specifically, cinnamic acid contained in Shea reduces skin inflammation and irritation as well as the compound, lupeol cinnimate which helps to reduce the incidence of skin mutations, having an anti-tumor effect. It is naturally rich in vitamins A, E and essential fatty acids. Shea butter also aids in the skin's natural collagen production and contains oleic, stearic, palmitic, and linolenic acids that protect and nourish the skin to prevent drying. It also allows our formula to become creamy with a soft and spreadable consistency after whipping.

Organic Mango Butter, Mangifera Indica, has been selected as another option of butter to be used alone or in combination with Organic Shea butter and Organic Cocoa butter. It is derived from the emulsified pit of the mango fruit which creates a semi-soft, creamy and ultra-hydrating effect on the skin. This can help any dryness associated with astringent properties of other components. Mango Butter is also used to alleviate itching which can be caused by shaving as well as transitioning to a new skin care product regimen. Mango butter is used in our formulation for the protectant qualities as well as nourishing and healing properties to more vulnerable skin as well as helps to enhance collagen production and promote skin elasticity.

Organic Cocoa Butter was the third alternative butter we have selected for our formulation to be used in conjunction with the Organic Mango and the Organic Shea butters, or to be used alone. When using alone, it is solid at room temperature and has a low melting point. We have chosen Cocoa butter as it soothes burns, rashes and infections-all of which can be present in the axillary region on a daily basis as a result of shaving, ingrown hairs and razor burn. It fights free radical damage which not only prevents harm, but provides a protective factor to health maintenance. As with the other butters we have incorporated, Cocoa butter is an excellent anti-inflammatory and moisturizer which makes it an ideal product for our purposes of a natural skin care product, deodorant.

Organic Apple Cider Vinegar is a natural mild astringent that reduces the incidence of sweat by constricting skin pores hence reducing body odor produced by apocrine glands in the underarm area. Apple cider vinegar is a highly acidic ingredient that will neutralizes the alkaline environment provided by sweat which in turn destroys the body odor-causing bacteria. Apple cider vinegar also prevents the formation and growth of bacteria along with flushing out toxins that causes body odor.

Sodium bicarbonate is used to neutralize and eliminate odor that are caused from bacteria and sweat in the axillary region. Pure sodium bicarbonate combats body odor while reacting with the ACV to help achieve a healthy and normal pH balance to reduce odor causing bacteria from being produced.

The Nopal Cactus, also known as the Prickly Pear Cactus is added to our product formulation and used to offset any irritation and soothe the underarm skin. The combination of Nopal Cactus and our organic butters and oils result in unmatched skin replenishing odor protection. Nopal Cactus contains Vitamin A offering superb anti-aging, antioxidant, and moisturizing capabilities. It accelerates cell renewal and stimulates fibroblast and collagen production in the skin. Nopal Cactus is high in antioxidants and phytochemicals that fight free radicals which can attack cells that can lead to dysplasia. An increasing number of studies are showing that topical application of the Nopal Cactus can disrupt carcinogenic pathways which is an opposite effect of most commercial antiperspirant/deodorants. As an increasing amount of research shows aluminum compounds (the main ingredient in antiperspirants) build up in mammary glands of breast tissue, adjacent to the application site as well as lymph node toxification. The Nopal cactus is one of the most betalain rich plants in existence-they are the pigments that make the Nopal cactus fruits the bright red color they are. Betalains contain a high amount of antioxidants as well as riboflavin which aids in tissue repair and helps protect skin from eczema and dermatitis. Phytochemicals are potent natural antioxidants that fight oxygenated molecules, aka free radicals. Nopal Cactus contains vitamin C to help stimulate cell turnover, reduce ingrown hairs as well as improving the quality of the skin by increasing elasticity. Vitamin K plays a key role in maintaining skin elasticity and helping to prevent signs of scarring in skin tissue, it also helps to reduce and regulate excess sebum (oil) production. Nopal cactus is underutilized in skin care products for its nutrients that are able to absorb through the skins largest organ as well as their vital disease prevention components that can successfully replace toxic alternatives in the current market.

Essential Oils are added to contribute to the fragrance of the product and the health promoting properties of our formula because of their antidepressant, stimulating, detoxifying, antibacterial, antiviral and calming effects. They are natural, safe and allow our product to encompass a therapeutic scent of the consumer's choice. The particles in essential oils come from distilling or extracting the different parts of plants, including the flowers, leaves, bark, roots, resin and peels. One drop of an essential oil can have powerful health benefits. They are created through the process of distillation which separates the oil and water-based compounds of a plant by steaming, essential oils are highly concentrated oils that have a strong aroma. By concentrating the oils of these plants, the essential oil separates the most powerful healing compounds of a plant into a single oil and is added to our formula for optimal health.

Food grade diatomaceous earth (DE) is a gentle powder that's also highly absorbent and it's almost entirely made of silica—an important component of human ligaments, cartilage, and musculature. DE is a naturally occurring soft, siliceous sedimentary rock that is easily crumbled into a fine white to off-white powder. Depending on the granularity, this powder can have an abrasive feel, similar to pumice powder, and has a low density as a result of its high porosity. It is an effective ingredient in natural deodorant because it can absorb twice its quantity in liquid, deodorizes unwanted smells, is known to draw out heavy metals which could potentially speed up the detoxification process from antiperspirant use and it is less alkaline than sodium bicarbonate therefore a great alternative for our formula also, when sodium bicarbonate is undesirable.

Organic Carnauba Wax is a natural wax that comes from the leaves of the Copernicia prunifera palm grown only in Brazil. The wax is obtained by beating the wax off of the dried palm fronds and then refining it for use. Carnauba wax consists of fatty acids and has a very high melting point of 82-86° C. (180-187° F.) which is why it is chosen as a stabilizer for our deodorant products that can be shipped to warmer climates. It is also a vegan wax and keeps our product cruelty free while free of toxins, hypoallergenic and an effective emollient.

This invention has many advantages. Our formulation of natural deodorant avoids the previously mentioned conflict and in fact, addresses and supports natural body functions such as perspiration and body odor while supporting skin condition. Our natural deodorant neutralizes odor causing bacteria as well as absorbing perspiration while using a completely natural formulation so our consumers have a product that is effective and will cause no adverse effects on their health for the short and long term. The combination of Nopal cactus and our organic butters and oils result in unmatched skin replenishing odor protection as a deodorant and helps absorb perspiration. Our unique formulation consists of a gentle and effective deodorant that eliminates the need for antiperspirant use as any perspiration produced with natural deodorant use contains fewer odor causing bacteria. Our product is composed of all organic, health promoting natural ingredients and specifically the unique addition of Opuntia Ficus Indica, or the Nopal Cactus chosen to promote and enhance skin health. Studies have shown that Opuntia ficus-indica, or Nopal Cactus can disrupt carcinogenic pathways when applied topically. Specifically, the topical anti-inflammatory effect of IGR (isorhamnetin diglycoside contained in Nopal Cactus) is mediated by the suppression of COX-2 (cyclooxygenase-2) activity and inhibition on the production of proinflammatory cytokines such as TNF-a (Tumor Necrosis Factor) and IL-6 (interleukin-6) production. These actions are an exciting advancement from conventional antiperspirant products. Our formulation incorporates the Nopal Cactus added during the cooling processes (to preserve the most optimal levels of enzymes) to our blend of organic oils and butters. Depending on consumer selection, the cactus is added along with Coconut Oil and a base of either Shea Butter, Mango Butter or Cocoa Butter to reduce any unwanted skin irritation, combat inflammatory responses from shaving, nicks or individualized reaction to commercial product use as well as enhance skin condition and cell renewal in the area it is applied to. As scientific studies on this widely underutilized plant continue to grow, one valuable piece of evidence has already emerged. The Nopal Cactus could be ground breaking in the field of disease prevention, especially when applied to the axillary region as an alternative to aluminum based compounds. The other ingredients selected for our effective formulation are EITHER sodium bicarbonate and apple cider vinegar or diatomaceous earth, arrowroot starch and essential oils. The ingredients produce odor control by reducing the type of bacteria that causes unwanted odors as well as sweat combating action through their natural pH balancing properties as well as their antiseptic properties. They inhibit negative effects on the beneficial flora or “probiotics” found naturally on healthy skin to maintain health and minimize any possibility of infection from harmful pathogens. The combination of sodium bicarbonate and Nopal Cactus results in a pH level closer to the body's natural level. This eliminates irritation which can result from using sodium bicarbonate on the skin.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A topical deodorant composition comprising:
Organic refined Shea butter;
Organic Nopal Cactus;
an ingredient selected from the group consisting of Sodium bicarbonate or Food Grade Diatomaceous Earth;
Arrowroot starch;
Organic Cocoa butter;
Organic Mango Butter;
Organic Carnauba Wax;
Organic Bees Wax;
Organic Coconut oil;
Organic Apple Cider Vinegar; and
pure essential oil extracts.

2. The topical deodorant composition of claim 1, wherein said refined Shea butter comprises from about 10.0% to about 30% by weight of said topical deodorant composition.

3. The topical deodorant composition of claim 1, wherein said sodium bicarbonate comprises from about 18.0% to about 25.0% by weight of said topical deodorant composition.

4. The topical deodorant composition of claim 1, wherein said arrowroot starch comprises from about 18% to about 25.0% by weight of said topical deodorant composition.

5. The topical deodorant composition of claim 1, wherein said cocoa butter comprises from about 10% to about 35% by weight of said topical deodorant composition.

6. The topical deodorant composition of claim 1, wherein said mango butter comprises from about 10.0% to about 25% by weight of said topical deodorant composition.

7. The topical deodorant composition of claim 1, wherein said apple cider vinegar comprises from about 0.03% to about 0.6% by weight of said topical deodorant composition.

8. The topical deodorant composition of claim 1, wherein said coconut oil comprises from about 0.8% to about 25% by weight of said topical deodorant composition.

9. The topical deodorant composition of claim 1, wherein said diatomaceous earth comprises from about 0.01% to about 10%

10. The topical deodorant composition of claim 1, wherein said nopal cactus comprises from about 8.0% to about 14% by weight of said topical deodorant composition.

11. The topical deodorant composition of claim 1, wherein said organic carnauba wax comprises 0.50%-20% by weight of said topical deodorant composition.

12. The topical deodorant composition of claim 1, wherein said organic bees wax comprises 0.50%-20% by weight of said topical deodorant composition.

13. The topical deodorant composition of claim 1, comprising:
27% unrefined Shea butter;
23% sodium bicarbonate;
25% arrowroot starch;
8.8% nopal cactus;
16% coconut oil;
0.10% apple cider vinegar; and
0.10% essential oil or combination of essential oils.

14. The topical deodorant composition of claim 1, comprising:
20% unrefined organic shea butter;
7% organic carnauba wax
23% sodium bicarbonate;
25% arrowroot starch;
8.8% nopal cactus;
16% coconut oil;
0.10% apple cider vinegar; and
0.10% essential oil or combination of essential oils.

15. The topical deodorant composition of claim 1, comprising:
27% organic cocoa butter;
23% sodium bicarbonate;
25% arrowroot starch;
8.8% nopal cactus;
16% coconut oil;
0.10% apple cider vinegar; and
0.10% essential oil or combination of essential oils.

16. The topical deodorant composition of claim 1, comprising:
25% organic cocoa butter;
3% food grade diatomaceous earth
6% organic carnauba wax
23% sodium bicarbonate;
25% arrowroot starch;
8.8% nopal cactus;
9% coconut oil;
0.10% apple cider vinegar; and
0.10% essential oil or combination of essential oils.

17. The topical deodorant composition of claim 1, comprising:
27% organic mango butter;
23% sodium bicarbonate;
25% arrowroot starch;
8.8% nopal cactus;
16% coconut oil;
0.10% apple cider vinegar; and
0.10% essential oil or combination of essential oils.

18. The topical deodorant composition of claim 1, comprising:
27% organic mango butter;
10% organic carnauba Wax
23% sodium bicarbonate;
22% arrowroot starch;
3% diatomaceous earth
8.8% nopal cactus;
6% coconut oil;
0.10% apple cider vinegar; and
0.10% essential oil or combination of essential oils.

19. The topical deodorant composition of claim 1, comprising:
10% unrefined shea butter;
10% organic mango butter
10% organic cocoa butter
23% sodium bicarbonate;
25% arrowroot starch;
8.8% nopal cactus;
16% coconut oil;
0.10% apple cider vinegar; and
0.10% essential oil or combination of essential oils.

20. The topical deodorant composition of claim 1, comprising:
9% unrefined shea butter;
9% organic mango butter
9% organic cocoa butter
3% diatomaceous earth
10% organic carnauba wax
23% sodium bicarbonate;
25% arrowroot starch;
8.8% nopal cactus;
6% coconut oil;
0.10% organic apple cider vinegar; and
0.10% essential oil or combination of essential oils.

21. A method of applying a topical deodorant composition of unrefined Shea butter, an ingredient selected from the group consisting of Sodium bicarbonate or Food Grade Diatomaceous Earth, Arrowroot Starch, Cocoa Butter, Mango Butter, pure essential oil; Nopal Cactus, Coconut oil, Apple Cider Vinegar, and optionally, Carnauba wax or Beeswax, said method comprising the step of applying said topical deodorant composition to an area of skin.

22. A process of manufacturing a topical deodorant composition, said process comprising:
mixing unrefined Shea butter, cocoa butter, coconut oil and/or mango butter into a composition;
heating said composition over hot water at approximately 100° F. to 120° F.;
stirring said composition until said composition is melted;
adding arrowroot starch and sodium bicarbonate into said composition;
stirring and heating said composition over hot water at about 100° F. to about 120° F. until said Arrowroot starch and said sodium bicarbonate are completely dissolved;
adding in Nopal Cactus after the composition has been completely dissolved and combining the Nopal Cactus with the composition until both the Nopal Cactus and composition are evenly distributed throughout thereby producing the final composition.

23. The method of claim 22, further comprising the step of:
placing the final composition in a glass jar.

24. The method of claim 22, further comprising the step of:
loading the final composition as a solid stick into a dispenser.

25. The method of claim 22, further comprising the step of:
placing the final composition in a squeezable tube.
